(19) United States
(12) Patent Application Publication
(10) Pub. No.: US 2024/0342468 A1
(43) Pub. Date: Oct. 17, 2024

(54) UNDER LENS DEVICE INCORPORATING HYALURONIC ACID

(71) Applicant: TWENTY TWENTY THERAPEUTICS LLC, South San Francisco, CA (US)

(72) Inventors: Daniel Patrick Barrows, Bloomfield, NY (US); Zidong Li, South San Francisco, CA (US); Fred Lee, South San Francisco, CA (US)

(21) Appl. No.: 18/633,625

(22) Filed: Apr. 12, 2024

Related U.S. Application Data
(60) Provisional application No. 63/459,420, filed on Apr. 14, 2023.

(51) Int. Cl. A61N 1/20; A61N 1/04; A61N 1/36; G02C 7/04; G16H 20/30

(52) U.S. Cl. CPC A61N 1/205; A61N 1/0476; A61N 1/36046; G02C 7/04; G16H 20/30

(57) ABSTRACT

An under-lid device (ULD), at least a portion of which is coated with a hydrogel comprising hydroxypropyl methylcellulose and/or hyaluronic acid, is described. Methods of using the ULD to treat dry eye, kits for the ULD, and methods of incorporating the tear retention polymers hydroxypropyl methylcellulose or hyaluronic acid into the hydrogel are also described.

### CROSS-REFERENCE TO RELATED APPLICATIONS

This application claims priority to U.S. Provisional Patent Application Ser. No. 63/459,420, filed Apr. 14, 2023, which is incorporated herein by reference.

### BACKGROUND

A large number of people have Dry Eye Disease (“DED”), which includes symptoms of intense pain, stinging eyes, foreign body sensation, light sensitivity, blurriness, increased risk of infection, and possible vision loss.

DED is characterized by insufficient tear volume on the ocular surface of a patient, which is generally caused by insufficient tear production or excessive tear evaporation. Insufficient tear volume results in tear hyperosmolarity, which causes inflammation and nerve damage and can lead to progressive loss of tear production and quality.

Dry-eye symptoms vary based on a variety of factors. For example, dry-eye symptoms vary throughout a day in response to diurnal physiological variations in tear pH, intraocular pressure, corneal sensitivity, visual sensitivity, and melatonin production. For instance, corneal sensitivity is often significantly greater in the evening than compared to the morning. Longer term variations in dry-eye symptoms can be related to use of systemic medications, chronic disease (e.g., diabetes), hormonal changes, and aging. Changes to a patient's environment also contribute to dry-eye symptom variations. For example, dry-eye symptoms can increase due to low humidity of air-conditioned offices, winter heating, computer use, phone use, allergens, and contact lenses.

Under-lid devices have been described for treating dry eye by stimulating tear production. However, a contributing factor for many dry eye patients is a decreased ability to maintain a robust tear film. Accordingly, there remains a need to provide a device that can be used to treat dry eye by both increasing tear production while helping to maintain a robust tear film.

### SUMMARY OF THE INVENTION

One component of dry eye disease is insufficient tear production, which has been addressed by some prior art under-lid devices by delivering an electrical stimulation to the lacrimal gland to trigger tear production. An additional contributing issue to dry eye is the inability to maintain a robust tear film. The present invention incorporates a tear retaining polymer such as hydroxypropyl methylcellulose or hyaluronic acid into the under-lid device to provide a combined effect of stimulating year production and maintaining a robust tear film in order to improve treatment of dry eye.

### BRIEF DESCRIPTION OF THE FIGURES

FIG. 1 is a perspective view of a therapeutic device for stimulating nerves in and around a patient's eye, according to aspects of the present disclosure.

FIG. 2 is an illustration of a periocular device to be worn around an eye, and proximate to a lacrimal gland, of a user, according to an example embodiment.

FIG. 3 is a diagrammatic illustration of the device of FIG. 2, according to an example embodiment.

### DETAILED DESCRIPTION OF THE INVENTION

The present invention provides an under-lid device (ULD), at least a portion of which is coated with a hydrogel comprising a tear retention polymer such as hydroxypropyl methylcellulose or hyaluronic acid. Methods of using the ULD to treat dry eye, kits for the ULD, and methods of incorporating the tear retention polymers hydroxypropyl methylcellulose or hyaluronic acid into the hydrogel are also provided.

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

Also herein, the recitations of numerical ranges by endpoints include all numbers subsumed within that range (e.g., 1 to 5 includes 1, 1.5, 2, 2.75, 3, 3.80, 4, 5, etc.).

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The terms “comprises,” “comprising,” “includes,” “including,” “having” and their conjugates mean “including but not limited to”.

The phrase “consisting essentially of” means that the composition or method may include additional ingredients and/or steps, but only if the additional ingredients and/or steps do not materially alter the basic and novel characteristics of the claimed composition or method.

The conjunctive phrase “and/or” indicates that either or both of the items referred to can be present.

A “subject,” as used herein, can be any animal, and may also be referred to as the patient. Preferably the subject is a vertebrate animal, and more preferably the subject is a mammal, such as a research animal (e.g., a mouse or rat) or a domesticated farm animal (e.g., cow, horse, pig) or pet (e.g., dog, cat). In some embodiments, the subject is a human.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses.

“Biocompatible” as used herein, refers to any material that does not cause injury or death to a subject or induce an adverse reaction in a subject when placed in contact with the subject's tissues. Adverse reactions include for example inflammation, infection, fibrotic tissue formation, cell death, or thrombosis. The terms “biocompatible” and “biocompatibility” when used herein are art-recognized and mean that the material is neither itself toxic to a subject, nor degrades (if it degrades) at a rate that produces byproducts at toxic concentrations, does not cause prolonged inflammation or irritation, or does not induce more than a basal immune reaction in the host.

As used herein, “treatment” means any manner in which the symptoms of a defect, condition, disorder, or disease, or any other indication, are ameliorated or otherwise beneficially altered.

### Coated Under-Lid Device

In one aspect, the present invention provides an under-lid device (ULD), at least a portion of which is coated with a hydrogel comprising a tear retaining polymer such as hydroxypropyl methylcellulose and/or hyaluronic acid. An under-lid device is an ophthalmological device that is designed to be positioned under an eyelid of the user. The under-lid device can be positioned partially or completely under the eyelid. Preferably, the under-lid device is an ophthalmological device for treating dry eye. Examples of under-lid devices include contact lenses and substrates (e.g., polymeric substrates) that are shaped so that they can be positioned stably under the lower eyelid or upper eyelid, and periocular rings. In some embodiments, the underlid device is a contact lens including an embedded electronic circuit that can be used to stimulate the lacrimal gland to increase tear production. The contact lens can be formed from a hydrogel material.

In some embodiments, the ULD comprises a polymer substrate. Preferably, the ULD comprises a biocompatible polymer substrate. Examples of biocompatible polymers include polymers include natural or synthetic polymers such as polystyrene, polylactic acid, polyketal, butadiene styrene, polymethylmethacrylate (“PMMA”), Parylene, Polyethylene terephthalate (“PET”), polyurethane, polyimide, polyamide, liquid crystal polymer, silicon-based polymers, silicone acrylate and the like. The polymer substrate may include a single layer of materials, or multiple layers of the same or different materials.

The ULD can include one or more electrodes and a microcontroller operably coupled to the electrodes, wherein the microcontroller is configured to activate the electrodes to stimulate a lacrimal gland of the user. The microcontroller can be included in the ULD, or it can be included in a remote controller that communicates with the ULD (e.g., through an antenna). The lacrimal gland is located within the orbit above the lateral end of the eye, where it continually releases fluid which cleanses and protects the eye's surface as it lubricates and moistens it.

In some embodiments, the ULD comprises polymeric substrate configured to be mounted or positioned under the eyelid of a subject (e.g., positioned in a patient's fornix). Such underlid devices can be shaped in such a way as to be positioned stably under the lower eyelid or upper eyelid, such as having a generally kidney-bean like shape. Such positioning allows the ULD to contact the tear film on the eye surface so that sensors within the ULD can determine if sufficient tear film is present. In some embodiments, the ULD has a thickness profile wherein the thicker regions of the thickness profile enhance on-eye stability of the ULD. Such devices are described by U.S. Pat. No. 11,191,462 and U.S. Patent Publication No. 2023/0011432, the disclosures of which are incorporated herein by reference.

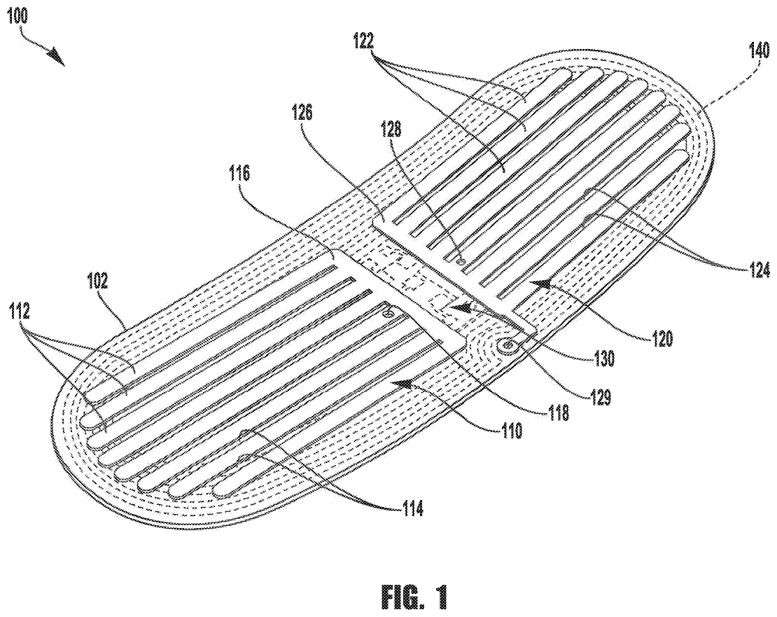

FIG. 1 is a perspective view of a ULD device 100 configured to be worn underneath a patient's eyelid (e.g., in the fornix) for stimulating nerves and/or the lacrimal gland in and/or around the patient's eye. The device 100 is flexible along at least one axis and is sized and shaped to fit under an eyelid. The device 100 includes electrodes 110, 120 configured to stimulate nerves and/or the lacrimal gland in and around the patient's eye, which may cause or enhance tear production, or other physiological responses to treat dry eye or other ophthalmic conditions.

The device 100 shown in FIG. 1 includes a first electrode 110 and a second electrode 120 disposed on a substrate 102. The first and second electrodes include an electrode spine 116 and electrode prongs 122. The device also includes a plurality of vias 118, 128, and 129, which provide points of electrical connection between the electrodes 110, 120, and the electronic components 130. An antenna 140 is disposed on opposing side of the substrate 102 and is configured to receive electromagnetic energy from a remote wireless device, and to harness the electromagnetic energy to produce electrical power for the components of the device 100. Electronic components 130 are also attached to the substrate 102 and in electrical communication with the antenna 140 and the electrodes 110, and 120.

In some embodiments, the ULD comprises a periocular ring configured to be worn on an eye of a user, the periocular ring having an opening, wherein a portion of the eye extends through the opening of the periocular ring when the periocular ring is worn on the eye of the user. A ring-shaped periocular neurostimulator for electrically stimulating the lacrimal gland to increase tear production has been described. See U.S. Pat. No. 11,376,432, the disclosure of which is incorporated herein by reference. Generally, the ring forms a circular or ring shape with an uninterrupted circumference or periphery. However, in some embodiments, a gap is formed within the ring to form a C shape. The periocular device can rest on the surface of the eye so no surgical procedures or implanted or implantable devices are needed for dry-eye therapy.

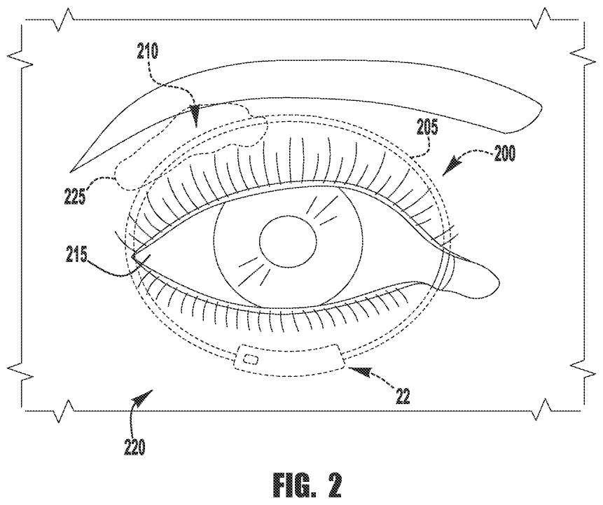

A periocular ring ULD 200, as illustrated in FIG. 2, is an example of a ring-shaped periocular device for neurostimulation and treating dry eye disease. The periocular ring ULD 200 generally includes a wearable band or ring 205 and a gland stimulator assembly 210. In some embodiments, the stimulator assembly 210 is considered part of the ring 205, as the stimulator assembly 210 is attached physically and electrically to the ring. The term “ring” used herein refers generally to a substantially circular shape but it is not so limited and may refer to an elliptical shape circumscribing, and spaced from, portions of the eye, such as the limbal ring. Generally, the ULD 200 is configured to encircle the front of an eye 215 of a user 220 in the ocular fornix area. For example, the ULD 200 may be worn outside the periphery of a user's iris, circumscribing the iris, and spaced radially away from the iris.

The periocular ring ULD 200 is positioned such that the gland stimulator assembly 210 is in close enough proximity to a lacrimal gland 225 of the user 220 to stimulate tear production when electrical signals are applied to the stimulator assembly. As is understood in the art, electrical stimulation of a lacrimal gland 225 is known to increase tear production. The gland stimulator assembly 210 comprises a plurality of electrodes 230 spaced along the periocular ring, and a microcontroller 235 operably coupled to the plurality of electrodes, and wherein the microcontroller is configured to activate the plurality of electrodes to stimulate a lacrimal gland of the user.

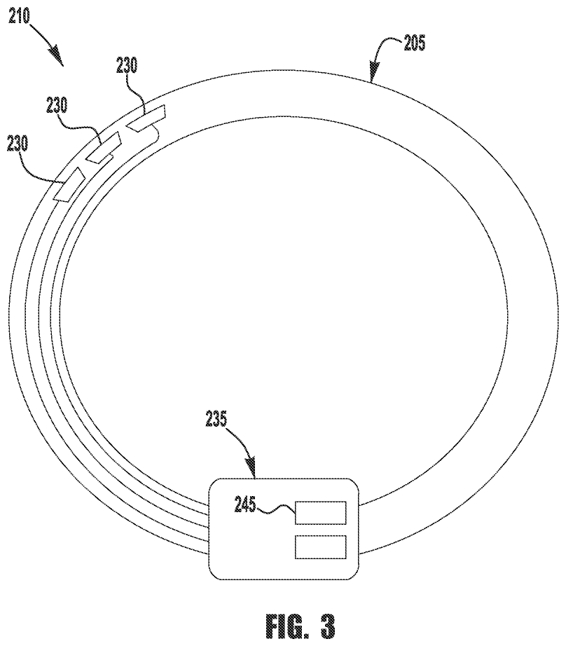

As illustrated in FIG. 3, the gland stimulator assembly 210 generally includes electrodes 230, a microcontroller 235, and a lead assembly 240 that operably couples the electrodes 230 to the microcontroller 235. Generally, the microcontroller 235 also includes, or is operably coupled to, a power source 245 and a memory 250. In some embodiments and when the power source 245 includes a battery, such as a lithium thionyl chloride cell, the battery has terminals connected to the input of a voltage regulator (not shown) that forms a portion of the microcontroller 235. The voltage regulator smooths the battery output and supplies power to the microcontroller 235, which controls the programmable functions of the stimulator assembly 210, including known stimulation parameters such as pulse amplitude (measured as current or voltage, e.g., 500 μA to 25 mA), pulse frequency, pulse width, and on-time and off-time of the output pulses supplied to the electrodes 230.

In some embodiments, the microcontroller 235 is programmable so that a patient profile can be stored in the memory 250 and used to regulate treatment. Using the patient profile, the microcontroller 235 modulates the electrical activity of the lacrimal gland to produce the treatment regimen applicable to the patient. Timing signals for the logic and control functions of the generator are provided by the memory 250.

Often, the polymer substrate, the gland stimulator assembly 210, and a sensor assembly (if present) are encapsulated in a soft flexible biocompatible material suitable for ocular wear. Examples of soft flexible biocompatible material includes polymeric material like PMMA, polyhydroxyethylmethacrylate (“polyHEMA”), silicone hydrogel, silicon based polymers (e.g., flouro-silicon acrylate), and silicone elastomer, or combinations thereof. Generally, the device 200 is sufficiently flexible to be bent and placed under the eyelid and the lower lid of the user 220.

At least a portion of the ULD is coated with a hydrogel. In some embodiments, at least a portion of an eye-contacting region of the ULD is coated with the hydrogel, while in other embodiments, at least a portion of a lid-contacting region of the ULD is coated with the hydrogel. A portion of the ULD can refer to a small region of the ULD or to the entire surface of the device. Accordingly, a portion of the ULD can refer to about 5%, about 10%, about 20%, about 30%, about 40%, about 50%, about 60%, about 70%, about 80%, about 90%, about 95%, or about 100% of the surface area of the device.

A hydrogel may be defined as a three-dimensional, hydrophilic or amphiphilic polymeric network capable of taking up large quantities of water. The networks are composed of homopolymers or copolymers, are insoluble due to the presence of covalent chemical or physical (ionic, hydrophobic interactions, entanglements) crosslinks. The crosslinks provide the network structure and physical integrity. Hydrogels exhibit a thermodynamic compatibility with water that allows them to swell in aqueous media.

In some embodiments, the hydrogel is prepared by crosslinking hydrophilic biopolymers or synthetic polymers. Examples of the hydrogels formed from physical or chemical crosslinking of hydrophilic biopolymers, include but are not limited to, hyaluronans, chitosans, alginates, collagen, dextran, pectin, carrageenan, polylysine, gelatin or agarose. Examples of hydrogels based on crosslinked synthetic polymers include but are not limited to (meth)acrylate-oligolactide-PEO-oligolactide-(meth)acrylate, poly(ethylene glycol) (PEO), poly(propylene glycol) (PPO), PEO-PPO-PEO copolymers (Pluronics), poly(phosphazene), poly(methacrylates), poly(N-vinylpyrrolidone), PL(G)A-PEO-PL(G)A copolymers, poly(ethylene imine), etc. See A. S Hoffman, Adv. Drug Del. Rev 43, 3-12 (2002). In some embodiments, the hydrogel is an alginate or polyacrylamide hydrogel.

In some embodiments, the hydrogel comprises a superporous hydrogel. A superporous hydrogel is a three-dimensional network of a hydrophilic polymer that is capable of absorbing a large amount of water in a very short period of time due to the presence of a large number of intereconnected microscopic pores. Methods of preparing superporous hydrogels are known to those skilled in the art. Mastropietro et al., Expert Opin Drug Deliv., 9(1):71-89 (2012). Superporous hydrogels generally have an average pore size of 50 to 100 μm. Use of a superporous hydrogel on the side of the device facing the eyelid can contribute to the comfort of the user. In some embodiments, the superporous hydrogel can be included as a layer between two layers of regular hydrogel.

The ULD is coated with a hydrogel comprising one or more tear retention polymers. A tear retention polymer is a biocompatible polymer that encourages tear retention in the eye. Examples of tear retention polymers include polymeric viscosity enhancing agents such as cellulosic polymers, hyaluronic acid, guar, polyethylene glyclol, and 2-methacryloyloxy ethyl phosphorylcholine. In some embodiments, the tear retention polymer is hydroxypropyl methylcellulose and/or hyaluronic acid.

In some embodiments, at least a portion of the ULD is coated with a hydrogel comprising a hyaluronic acid derivative. Hyaluronic acid derivatives are hyaluronic acid polymers that have been modified to include an additional chemical group. Examples of hyaluronic acid derivatives include hyaluronic acid alkyl derivatives. An example of a specific hyaluronic acid derivative is methacrylated hyaluronic acid.

### Methods of Treating Dry Eye

Another aspect of the invention provides a method of treating dry eye in a subject in need thereof. The method includes positioning an under-lid device (ULD) between an eyelid and an eye of the subject, wherein at least a portion of the ULD is coated with a hydrogel comprising a tear retaining polymer such as hydroxypropyl methylcellulose and/or hyaluronic acid, and activating the ULD to stimulate a lacrimal gland of the user. The lacrimal gland of the subject can be stimulated by various methods such as heat, motion, or electric current.

An advantage of the method of the invention is that it provides two types of treatment of dry eye disease; namely, stimulation of tear formation from the lacrimal gland, and improved tear retention through release of the tear retaining polymer (e.g., hyaluronic acid) in the eye. In some embodiments, the ULD includes a gland stimulator assembly that includes a sensor and/or microprocessor that stimulates the lacrimal gland based on time or indicators of dry eye, taking into account a patient profile. Likewise, a sensor (e.g., an osmolarity sensor) or timing can be used to determine if additional tear retaining polymer should be released into the eye.

Dry eye disease (also known as dry eye syndrome or keratoconjunctivitis sicca is characterized by insufficient tear volume on the ocular surface of a patient. Symptoms include irritation, redness, discharge, and easily fatigued eyes. Symptoms can be worsened by activities in which the rate of blinking is reduced due to prolonged use of the eyes such as prolonged reading, computer usage (computer vision syndrome), driving, or watching television. Dry eye disease can be caused by a variety of different factors, including contact lens use, meibomian gland dysfunction, pregnancy, Sjögren syndrome, vitamin A deficiency, omega-3 fatty acid deficiency, LASIK surgery, and certain medications. Various diagnostic tests are available to detect and/or characterize dry eye disease, including slit lamp examination, Schirmer's test, the tear breakup time (TBUT) test, and lactoferrin level analysis.

The method of treatment includes the use of all of the various embodiments of the ULD for treating dry eye described herein. In some embodiments, the ULD comprises one or more electrodes and a microcontroller operably coupled to the of electrodes, and wherein the microcontroller is configured to activate the electrodes to stimulate a lacrimal gland of the user. In further embodiments, the ULD comprises a periocular ring configured to be worn on the eye of the user. The periocular device rests on the surface of the eye, so no surgical procedures or additional implanted or implantable devices are needed to provide dry-eye therapy. The ULD is insertable in the eye (e.g., the periocular space) and can be removable for cleaning and/or recharging. Thus, insertion and removal of the ULD can be performed without the need for surgery. In some instances, the user can insert and remove the ULD themselves, such as at home.

In some embodiments, at least a portion of the ULD is coated with a hydrogel comprising hyaluronic acid. In further embodiments, the hydrogel of the ULD comprises a superporous hydrogel. In yet further embodiments, the ULD stimulates the lacrimal gland of the user based on a temporal model associated with the subject, which can be used as part of the programmable functions of the ULD. The temporal model could detail a treatment program that is cycled daily, weekly, monthly, and/or seasonally. In some instances, the temporal model is based on a monitored eye condition of the patient/user, a charting of perceived symptoms by the patient/user, and/or a generic temporal model based on patient: age, sex, weight, geographical location, profession, activity level, or any combination thereof.

The method of use can vary depending on the wear modality of the device. For daily disposable devices it may be sufficient to initially provide the device with enough tear retaining polymer (e.g., hyaluronic acid) for single wear duration. However, if the device is intended for use over several days, it can be designed so that the tear retaining polymer (e.g., hyaluronic acid) can be re-charged when the device is stored overnight.

In some embodiments, the method of treating dry eye also includes treatment with one or more additional dry eye medications. The medication can be included in the ULD (e.g., within the hydrogel) or can be administered separately. Medications for treating dry eye disease include: Mucosta® (rebamipide ophthalmic suspension 2%, Otsuka Pharmaceutical, Tokyo, Japan) and Diquas® (diquafosol ophthalmic solution 3%, Santen Pharmaceutical, Osaka, Japan) in Asia; Xiidra® (lifitegrast ophthalmic solution 5.0%, Shire, Lexington, KY, USA) and Restasis® (Cyclosporine ophthalmic emulsion 0.05%, Allergan, Irvine, CA, USA) in North America; and Ikervis® (CsA cationic emulsion, Santen Pharmaceutical, Osaka, Japan) in Europe. In addition, Cequa™ (ananomicellar formulation of CsA 0.09%, Sun Pharmaceuticals, Mumbai, India) has been used in the US since 2018. Cequa™ enhances lacrimal fluid production in patients with dry eye disease.

### Kits for Treating Dry Eye

Another aspect of the invention provides a kit for treating dry eye. The kit includes an under-lid device (ULD) wherein at least a portion of the ULD is coated with a hydrogel; a hydrating solution including tear retaining polymer such as hydroxypropyl methylcellulose or hyaluronic acid; and a package for both the ULD and the hydrating solution. In some embodiments, the package includes a storage space for the hydrating solution that is configured to allow storage of the ULD within the hydrating solution. This kit can be used to uptake the tear retention polymer (e.g., hyaluronic acid) into the hydrogel during hydration and storage. The tear retention polymer (e.g., hyaluronic acid) can be provided in powder form which readily dissolves in an aqueous solution (e.g., the hydrating solution).

The kit can include any of the various embodiments of the ULD for treating dry eye described herein. In some embodiments, the hydrating solution includes hyaluronic acid. In further embodiments, the hydrogel is a superporous hydrogel. In some embodiments, the kit can include a device to provide positive or negative pressure to the storage space for the hydrating solution to facilitate uptake of the tear retention polymer.

### Methods of Incorporating Tear Retention Polymers

Another aspect of the invention provides a method of incorporating a tear retention polymer into an under-lid device (ULD) wherein at least a portion of the ULD is coated with a hydrogel comprising the tear retention polymer. The tear retention polymer can be incorporated into the hydrogel either by soaking the hydrogel in a hydrating solution including the tear retention polymer or by including the tear retention polymer during preparation of the hydrogel from hydrogel monomers. In some embodiments, the tear retention polymer is hydroxypropyl methylcellulose or hyaluronic acid.

For example, for embodiments in which at least a portion of the ULD is coated with a hydrogel comprising hyaluronic acid, the hyaluronic acid can be incorporated into the hydrogel by soaking the hydrogel in a hydrating solution including the hyaluronic acid or by including the hyaluronic acid during preparation of the hydrogel from hydrogel monomers.

The method can be used to incorporate tear retention polymer into any of the various embodiments of the ULD for treating dry eye described herein. In some embodiments, the tear retention polymer is hyaluronic acid. In further embodiments, the hydrogel is a superporous hydrogel.

In some embodiments, the tear retention polymer is incorporated into the hydrogel by soaking the hydrogel in a hydrating solution including the tear retention polymer. This method can be used to uptake tear retaining polymer into the hydrogel during hydration and storage of the ULD, as may occur if the ULD is removed and stored at the end of the day. The hydrating solution is an aqueous solution that is biocompatible and will provide water for re-hydrating the hydrogel. In some embodiments, the hydrating solution is a saline solution. Additional components of the hydrating solution can include those typically found in saline contact solution, such as polyquaternium, boric acid, propylene glycol, hydrogen peroxide, polaminopropyl biguanide, and sodium chloride. Only a small amount of the tear retaining polymer need be present in the hydrating solution. For example, the solution can include from 0.005% to 0.1%, or about 0.01% by weight of the tear retention polymer in the hydration solution.

In some embodiments, the tear retention polymer (e.g., hyaluronic acid) is included during preparation of the hydrogel from hydrogel monomers. When this is done, the tear retention polymer does not crosslink, but rather becomes entrained throughout the hydrogel matrix. Upon swelling of the hydrogel, the tear retention polymer will be released from the hydrogel. Those skilled in the art can readily determine the monomers from which a hydrogel is prepared. Examples of hydrogel monomers include dihydroxy methacrylates, methacrylic acid, acrylamides, and poly(ethylene glycol).

In some embodiments, a superporous hydrogel is used. A variety of methods can be used to prepare a superporous hydrogel. In some embodiments, the pores of the superporous hydrogel are prepared using micronized sucralose or sodium chloride as a porisogen. In other embodiments, a gas is bubbled through the hydrogel during crosslinking to form the pores of the superporous hydrogel. In other embodiments, a bicarbonate foaming agent can be used, where the foaming agent reacts with a foaming aid (e.g., an organic acid to generate carbon dioxide gas, which forms pores. The pores present in the superporous hydrogel can provide a reservoir for tear retention polymer within the hydrogel. Chavda et al. (Int J Pharm Investig., 2(3): 134-139 (2012)), the disclosure of which is incorporated by reference, describe the preparation of superporous hydrogels based on a variety of different polymers.

The complete disclosure of all patents, patent applications, and publications, and electronically available materials cited herein are incorporated by reference. Any disagreement between material incorporated by reference and the specification is resolved in favor of the specification. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An under-lid device (ULD), at least a portion of which is coated with a hydrogel comprising hydroxypropyl methylcellulose and/or hyaluronic acid.

2. The ULD of claim 1, wherein the ULD comprises a polymer substrate.

3. The ULD of claim 1, wherein the hydrogel comprises a superporous hydrogel.

4. The ULD of claim 1, wherein at least an eye-contacting region of the ULD is coated with the hydrogel.

5. The ULD of claim 1, wherein at least a portion of the ULD is coated with a hydrogel comprising hyaluronic acid.

6. The ULD of claim 1, wherein the ULD comprises one or more electrodes and a microcontroller operably coupled to the of electrodes, and wherein the microcontroller is configured to activate the electrodes to stimulate a lacrimal gland of the user.

7. The ULD of claim 1, wherein the ULD comprises a periocular ring configured to be worn on an eye of a user, the periocular ring having an opening, wherein a portion of the eye extends through the opening of the periocular ring when the periocular ring is worn on the eye of the user.

8. A method of treating dry eye in a subject in need thereof, comprising positioning an under-lid device (ULD) between an eyelid and an eye of the subject, wherein at least a portion of the ULD is coated with a hydrogel comprising hydroxypropyl methylcellulose and/or hyaluronic acid, and activating the ULD to stimulate a lacrimal gland of the user.

9. The method of claim 8, wherein the ULD comprises one or more electrodes and a microcontroller operably coupled to the of electrodes, and wherein the microcontroller is configured to activate the electrodes to stimulate a lacrimal gland of the user.

10. The method of claim 8, wherein the ULD comprises a periocular ring configured to be worn on the eye of the user.

11. The method of claim 8, wherein at least a portion of the ULD is coated with a hydrogel comprising hyaluronic acid.

12. The method of claim 8, wherein the hydrogel of the ULD comprises a superporous hydrogel.

13. The method of claim 8, wherein the ULD stimulates the lacrimal gland of the user based on a temporal model associated with the subject.

14. A kit for treating dry eye, comprising:
An under-lid device (ULD) wherein at least a portion of the ULD is coated with a hydrogel;
a hydrating solution including hydroxypropyl methylcellulose and/or hyaluronic acid; and
a package for both the ULD and the hydrating solution.

15. The kit of claim 14, wherein the ULD comprises a periocular ring configured to be worn on an eye of a user, the periocular ring having an opening, wherein a portion of the eye extends through the opening of the periocular ring when the periocular ring is worn on the eye of the user.

16. The kit of claim 14, wherein the hydrating solution includes hyaluronic acid.

17. The kit of claim 14, wherein the hydrogel is a superporous hydrogel.

18. The kit of claim 14, wherein the package includes a storage space for the hydrating solution that is configured to allow storage of the ULD.

19. A method of incorporating a tear retention polymer into an under-lid device (ULD) wherein at least a portion of the ULD is coated with a hydrogel comprising the tear retention polymer, wherein the tear retention polymer is incorporated into the hydrogel by soaking the hydrogel in a hydrating solution including the tear retention polymer or by including the tear retention polymer during preparation of the hydrogel from hydrogel monomers, wherein the tear retention polymer is hydroxypropyl methylcellulose or hyaluronic acid.

20. The method of claim 19, wherein the ULD comprises one or more electrodes and a microcontroller operably coupled to the of electrodes, and wherein the microcontroller is configured to activate the electrodes to stimulate a lacrimal gland of the user.

21. The method of claim 19, wherein the ULD comprises a periocular ring configured to be worn on an eye of a user, the periocular ring having an opening, wherein a portion of the eye extends through the opening of the periocular ring when the periocular ring is worn on the eye of the user.

22. The method of claim 19, wherein the tear retention polymer is incorporated into the hydrogel by soaking the hydrogel in a hydrating solution including the tear retention polymer.

23. The method of claim 19, wherein the tear retention polymer is included during preparation of the hydrogel from hydrogel monomers.

24. The method of claim 19, wherein at least a portion of the ULD is coated with a hydrogel comprising hyaluronic acid, wherein hyaluronic acid is incorporated into the hydrogel by soaking the hydrogel in a hydrating solution including the hyaluronic acid or by including the hyaluronic acid during preparation of the hydrogel from hydrogel monomers.

25. The method of claim 19, wherein the hydrogel is a superporous hydrogel.

26. The method of claim 25, wherein the pores of the superporous hydrogel are prepared using micronized sucralose or sodium chloride as a porisogen.

27. The method of claim 25, wherein gas is bubbled through the hydrogel during crosslinking to form the pores of the superporous hydrogel.
